(19) United States
(12) Patent Application Publication
(10) Pub. No.: US 2025/0174343 A1
(43) Pub. Date: May 29, 2025

(54) ADVANCED ASSISTANT AND USER INTERFACE FOR SPINAL CORD STIMULATION SELF-PROGRAMMING

(71) Applicant: Boston Scientific Neuromodulation Corporation, Valencia, CA (US)

(72) Inventors: Ismael Huertas Fernandez, Seville (ES); Que T. Doan, West Hills, CA (US)

(73) Assignee: Boston Scientific Neuromodulation Corporation, Valencia, CA (US)

(21) Appl. No.: 18/956,881

(22) Filed: Nov. 22, 2024

Related U.S. Application Data
(60) Provisional application No. 63/603,485, filed on Nov. 28, 2023.

(51) Int. Cl. G16H 40/63

(52) U.S. Cl. CPC G16H 40/63

(57) ABSTRACT

Methods and systems for reprogramming an implantable medical device. A reprogramming mode may be initiated in an application running on a patient remote control. In the reprogramming mode, one or more trial programs may be displayed on a user interface. The one or more trial programs may be displayed in response to receiving an initial patient efficacy rating below a predetermined threshold. The one or more trial programs may be calibrated. The implantable medical device may be successively operated in accordance with the parameters of each trial program. A patient efficacy rating may determine if any of the trial programs meet a predetermined acceptable pain management standard.

### CROSS REFERENCE TO RELATED APPLICATIONS

This application claims the benefit of U.S. Provisional Patent Application Ser. No. 63/603,485, filed on Nov. 28, 2023, and titled ADVANCED ASSISTANT AND USER INTERFACE FOR SPINAL CORD STIMULATION SELF-PROGRAMMING, which is related to copending U.S. Provisional Patent Application Ser. No. 63/603,490, filed on Nov. 28, 2023, and titled AUTOMATIC PROGRAM GENERATOR FOR SPINAL CORD STIMULATION, the disclosures of which are incorporated herein by reference.

### TECHNICAL FIELD

The disclosure is directed to programming and controlling spinal cord stimulation systems. More particularly, the disclosure is directed to methods and systems for patient reprogramming of spinal cord stimulation systems.

### BACKGROUND

Implantable and/or wearable stimulations systems for the treatment of various diseases and disorders of the neurological system have proven effective in a wide variety of ways. For example, spinal cord stimulation (SCS) systems are accepted treatments for chronic pain syndromes. An SCS system typically includes an Implantable Pulse Generator (IPG). The IPG is coupled to tissue-stimulating electrodes carried on the distal end of one or more electrode leads that are implanted near the spinal column. The proximal ends of the one or more leads are tunneled through the patient's tissue to a location such as the buttocks where the IPG is implanted. The proximal ends of the leads are coupled to the IPG to provide electrical stimulation from the electrodes to alleviate a patient's symptoms, often to treat pain.

Initial programming of the IPG may be completed by a clinician or a specially trained employee of the system manufacturer with physician instructions or guidance. Additional programming of the system may be done after the initial programming when the system fails to provide desired pain relief. Many programming sessions that occur after the initial programming may result in small changes to the stimulation parameters or simply re-education of the patient. New and alternative methods and devices which guide or allow the patient to perform limited reprogramming of the IPG may be desired.

### SUMMARY

The disclosure is directed to several alternative designs, materials and methods of manufacturing medical device structures and assemblies.

In a first example, a method for reprogramming an implantable medical device may comprise entering a reprogramming mode in an application running on a patient remote control, wherein the patient remote control includes a user interface. When in said reprogramming mode, the method may further comprise displaying on the user interface of the patient remote control one or more trial programs, wherein the one or more trial programs are displayed in response to receiving an initial patient efficacy rating of a current program below a predetermined threshold, calibrating the one or more trial programs, successively operating an implantable medical device in accordance with a set of parameters of each trial program of the one or more trial programs over a predetermined time period, receiving a patient efficacy rating for each trial program of the one or more trial programs, and determining if at least one trial program of the one or more trial programs meets a predetermined acceptable pain management standard.

Alternatively or additionally to any of the examples above, in another example, the reprogramming mode may be entered via an assistant. Alternatively or additionally to any of the examples above, in another example, the initial patient efficacy rating may be received in response to entering the reprogramming mode.

Alternatively or additionally to any of the examples above, in another example, the reprogramming mode may be entered in response to receiving a patient input at the user interface of the patient remote control. Alternatively or additionally to any of the examples above, in another example, the initial patient efficacy rating may be received during routine monitoring of the implantable medical device.

Alternatively or additionally to any of the examples above, in another example, the application may be configured to enter the reprogramming mode in response to the initial patient efficacy rating. Alternatively or additionally to any of the examples above, in another example, the method may further comprise selecting a trial program that meets the predetermined pain management standard. Alternatively or additionally to any of the examples above, in another example, the method may further comprise transmitting the selected trial program to the implantable medical device.

Alternatively or additionally to any of the examples above, in another example, the one or more trial programs may be selected from a plurality of anatomical based programs or paresthesia-based programs based off of a reference program. Alternatively or additionally to any of the examples above, in another example, calibrating the one or more trial programs may comprise increasing a stimulus until a paresthesia response is felt for each trial program of the one or more trial programs.

Alternatively or additionally to any of the examples above, in another example, calibrating the one or more trial programs may be performed in one or more postures. Alternatively or additionally to any of the examples above, in another example, calibrating the one or more trial programs may determine a therapeutic amplitude for each trial program of the one or more trial programs. Alternatively or additionally to any of the examples above, in another example, if at least one trial program of the one or more trial programs fails to meet a predetermined acceptable pain management standard one or more additional trial programs may be presented.

Further illustrative examples may take the form of a remote control configured for reprogramming an implantable medical device, remote control comprising a memory, a user interface for receiving inputs from and providing instructions or information to a patient, and a processor including a communication circuit for communicating with the implantable medical device, the memory storing in non-transitory manner instructions for performing the preceding methods to reprogram an implantable medical device.

In another example, a non-transitory computer-readable medium having instructions stored thereon that when executed by a patient remote control may be configured to receive an initial patient efficacy rating of a current program, display one or more trial programs, guide a patient through a calibration process of the one or more trial programs, successively operate an implantable medical device in accordance with a set of parameters of each trial program of the one or more trial programs over a predetermined time period, receive a patient efficacy rating for each trial program of the one or more trial programs, and determine if at least one trial program of the one or more trial programs meets a predetermined acceptable pain management standard.

Alternatively or additionally to any of the examples above, in another example, if at least one trial program of the one or more trial programs meets the predetermined acceptable pain management standard, receive a selection of a trial program.

Alternatively or additionally to any of the examples above, in another example, the non-transitory computer readable medium may be further configured to transmit the selected trial program to an implantable medical device.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 shows components of an illustrative neuromodulation system;

FIG. 2 shows an illustrative pulse generator and lead system;

FIG. 3 shows a spinal cord stimulation system as implanted in a patient;

FIG. 4 is a block diagram of an illustrative patient remote control;

FIG. 5 is an illustrative flow chart of an illustrative method for reprogramming an IPG; and

FIGS. 6A-6F depict illustrative displays that may be presented to the patient via the patient remote control during a reprogramming procedure

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

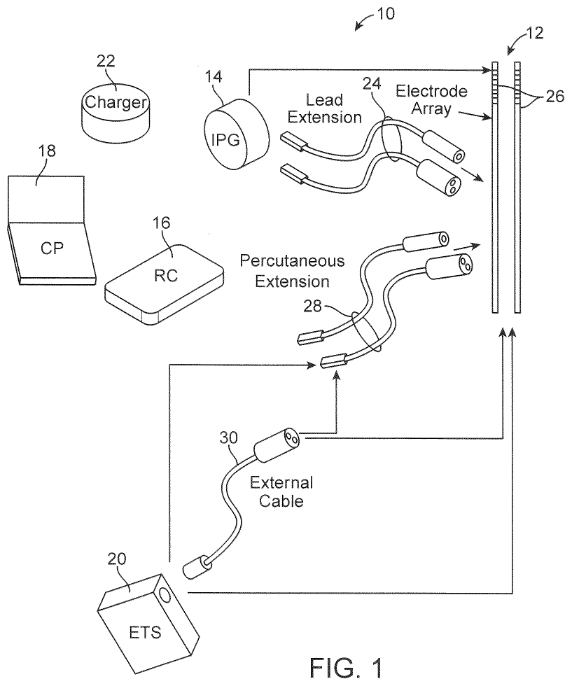

FIG. 1 shows a system 10 for providing neurological therapy, which may be used, for example, as spinal cord stimulation (SCS), deep brain stimulation (DBS), peripheral nerve stimulation (PNS), or functional electrical stimulation (FES). The system 10 includes leads 12 configured for coupling to an implantable pulse generator (IPG) 14. The IPG 14 may communicate with one or more of a patient remote control (RC) 16, a clinician programmer (CP) 18, and/or a charger 22. An external testing system (ETS) 20 may also be provided for testing therapy parameters prior to implantation of the IPG 14, using percutaneous extensions 28 and, as needed, an external cable 30 to couple to the leads 12. The ETS 20 may be used postoperatively during a trial period or testing procedure prior to permanent IPG 14 implantation. If needed, lead extensions 24 may be used to couple the IPG 14 to the leads 12.

As shown in FIG. 1, the leads 12 may include arrays of electrode contacts on linear leads 26; in other examples, paddle leads may also be used. One, two or even four leads 12 may be provided, with up to 32 contacts on the leads 12. An additional contact in the form of the housing of IPG 14 may be available in modern systems. More or fewer contacts on more or fewer leads may be provided depending the particular system.

The IPG 14 can couple directly to the leads 12 or may be coupled via the lead extensions 24, depending on the positioning of each element as implanted. The IPG 14 may include a rechargeable battery and charging coil to allow recharging when placed in proximity to the charger 22. Alternatively, the IPG 14 may use a non-rechargeable battery and omit a charging coil and charger 22 from the system. In some examples, the IPG 14 may be externally powered and omits a battery entirely.

The CP 18 can be used by a physician to manipulate the outputs of the IPG 14 and/or ETS 20. For example, the CP 18 can be used by the physician to define a therapy regimen or program for application to the patient. The CP 18 may be a custom device and/or may take the form of a laptop or tablet computer, for example and without limitation. Multiple programs may be facilitated and stored by the IPG 14 or ETS 20; in some examples, the RC 16 may store the programs to be used. Communication amongst the IPG 14, RC 16, CP 18, ETS 20 and charger 22 may use any suitable protocol such as wireless RF telemetry, Medradio, inductive communication, Bluetooth, etc.

The RC 16 may be used by a patient to enable or disable therapy programs, to select between available programs, and/or to modify the programs that are available for use. For example, in some embodiments a patient may use the RC 16 to activate a stored program and then manipulate therapy by increasing or decreasing therapy strength and/or changing therapy location, within limits set by the physician. The RC 16 may be a custom device, or may be, for example, a smartphone or tablet having an application thereon for use with the medical device system 10.

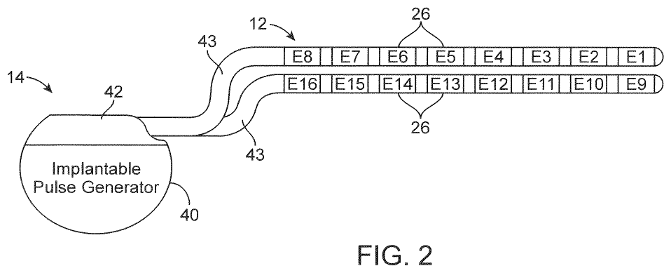

FIG. 2 shows an implantable stimulator and leads. As shown in the closer detail here, the IPG 14 may include a canister 40 and header 42. The canister 40 is conductive in most examples, using biocompatible materials such as titanium and/or stainless steel, for example, to allow use as an electrode when implanted. The header 42 allows removable connection to the lead 12, which in this example may have a bifurcation or yoke allowing two segments 43 to extend therefrom, to two arrays 26 at the distal end of the lead 12. A common structure for securing the leads 12 is the use of a setscrew in a port in the header 42. The electrode arrays 26 can be numbered as shown to facilitate case of understanding when programming, with, for example, one array marked electrodes E1 to E8 on one of the leads 43, with E1 being distal-most. Other conventions may be used.

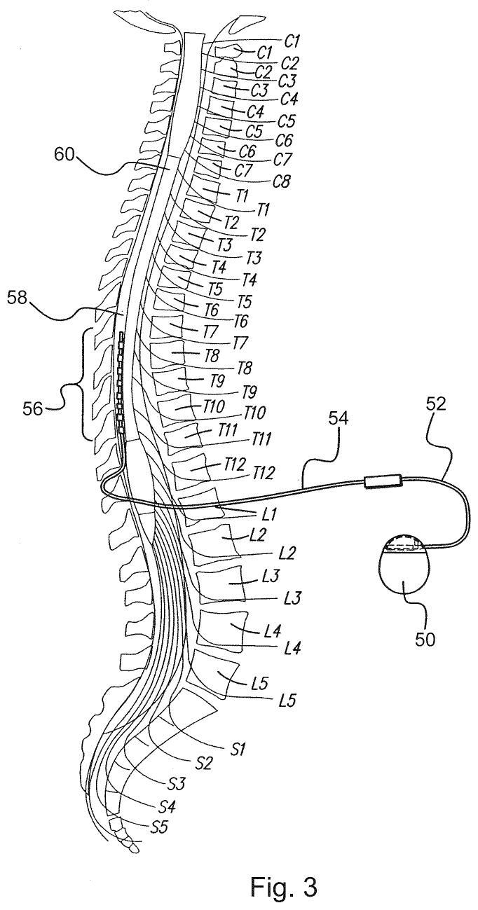

FIG. 3 shows an illustrative spinal cord stimulation system as implanted. In this example, an IPG 50 may be placed near the buttocks or in the abdomen of the patient, with or without a lead extension 52 for coupling to the lead(s) 54 that enter the spinal column. Region 56, at about the level of the lower thoracic or upper lumbar vertebrae may serve as an entry point to the spinal column, where the distal end of the lead 54 with an electrode array may be placed close to the spinal cord 58. Other locations for the IPG 50 and/or lead 54 may be used.

The standard approach to therapy in systems similar to those shown in FIGS. 1-3 has been that the IPG 14, 50, or ETS 20, may offer current controlled or voltage-controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

The leads 54, as shown in FIG. 3, are often placed in the thoracic region of the spinal column. It is contemplated that the positioning of the leads 54 may be determined based on the region where pain management is desired.

The initial programming of the IPG 14 may be completed by a clinician or a specially trained employee of the manufacturer of the system 10 using the CP 18. Further, additional programming of the system 10 may be done after the initial programming when the system 10 fails to adequately administer pain relief or paresthesia. Many programming sessions that occur after the initial programming may result in small changes to the stimulation parameters or simply re-education of the patient. A system which guides the patient through reprogramming of the IPG 14 is desired. The following examples show systems and methods for guiding a patient through reprogramming the IPG 14.

The IPG 14 may be capable of storing multiple different stimulation programs. During the initial programming of the IPG 14, at least one reference paresthesia program may be defined and stored. The electric field center coordinate of the reference program may be used to generate additional anatomical based programs or paresthesia-based programs for the patient to test. For example, upon definition of the reference program, additional anatomical based programs or paresthesia-based programs may be populated for use by a reprogramming assistant provided at the RC 16.

Copending U.S. Provisional Patent Application Ser. No. 63/603,490, filed on Nov. 28, 2023 and titled AN AUTOMATIC PROGRAM GENERATOR FOR SPINAL CORD STIMULATION, which is incorporated herein by reference, describes various examples of programs that may be generated based on a reference program and/or patient anatomy and prior tested program data. These may include anatomical-based programs in which different electrode sets are tested/used to determine how the patient responds to such programs. Anatomical-based programs may be updated over time to focus therapy testing and utilization on areas shown to provide patient benefits. These may also include paresthesia-based or reference-program-based test programs, which can be determined by identifying, for the reference program, a central point of stimulation (CPS), and then varying the CPS for different test programs. The CPS may be, for example, the mathematical center of output currents or voltages, with reference to either anodic outputs or cathodic outputs, or even calculated by reference to both anodes and cathodes. The copending application provides further examples and explanations.

It is contemplated that each time the reference program is defined (or redefined), the additional anatomical based programs or paresthesia-based programs may be repopulated based on the reference program. The programs can be generated by the CP during a following session with the patient, or generated by an RC later on in response to a patient indicating a lack of efficacy of one or more therapy programs, or generated remotely, if desired, using a remote server connected to the IPG, for example, via a bedside monitor (not shown) or the RC. These additional programs may be considered to be rescue programs for use when the initial program or reference program is no longer providing adequate pain relief. For any of these sources, the rescue programs may be stored in a memory of the RC 16 and/or the IPG 14.

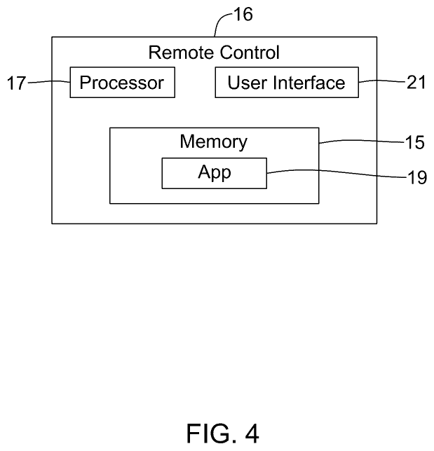

FIG. 4 is a block diagram of an illustrative RC 16. As noted above, the RC 16 may be a custom device provided to and used by the patient or may be an application (app) loaded onto the patient's smartphone, tablet computer, laptop, or the like. In some examples, the RC 16 may be a client device carried or worn by a patient. The RC 16 may include a user interface 21 including a display and a means for receiving user input (e.g., touch screens, buttons, keyboards, etc.). The RC 16 can include one or more software applications (e.g., apps) 19 stored within a memory 15 of the RC 16 that can define and/or control communications between the RC 16, the IPG 14 and/or other devices. In some cases, the RC 16 may communicate with the IPG 14, and/or other devices via a remote or cloud server. As noted above, the RC 16 may also include a communications circuit (or multiple communications circuit) for using infrared, cellular, WiFi, Bluetooth, Medradio or other modality to communicate with the IPG and/or other system components or a remote server.

The illustrative RC 16 includes a memory 15 and a processor 17. The processor 17 is configured to execute executable instructions stored in the memory 15 to perform various tasks. Data may also be stored in the memory 15 to be used in executing the instructions. The memory 15 can be any type of non-transitory storage medium that can be accessed by the processor 17 to perform various examples of the present disclosure. For example, the memory 15 can be a non-transitory computer readable medium having computer readable instructions (e.g., computer program instructions) stored thereon that are executable by the processor 17. The execution of the computer readable instructions may result in the transmittal of a program to the IPG 14 which in turn delivers therapy to the patient in accordance with the control parameters of the program. In some cases, the program may be temporarily operated in a trial capacity. The processor may be understood as including auxiliary circuitry to the processor including, for example, a power source (a rechargeable battery for example), communication circuitry as described above, and any suitable further analog or digital circuitry, including application-specific integrated circuits, amplifiers, digital signal processors, etc. that may be used in commercially known patient RC devices, as well as smartphones, tablet computers, etc. that may serve as an RC.

The memory 15 can be volatile or nonvolatile memory. The memory 15 can also be removable (e.g., portable) memory, or non-removable (e.g., internal) memory. For example, the memory 15 can be random access memory (RAM) (e.g., dynamic random access memory (DRAM) and/or phase change random access memory (PCRAM)), read-only memory (ROM) (e.g., electrically erasable programmable read-only memory (EEPROM) and/or compact-disc read-only memory (CD-ROM)), flash memory, a laser disc, a digital versatile disc (DVD) or other optical storage, and/or a magnetic medium such as magnetic cassettes, tapes, or disks, among other types of memory.

Further, although memory 15 is illustrated as being located within the RC 16, embodiments of the present disclosure are not so limited. For example, memory 15 can also be located internal to another computing resource (e.g., enabling computer readable instructions to be downloaded over the Internet or another wired or wireless connection). This may include the use of a remote computer or server.

In addition to, or in place of, the execution of executable instructions, various examples of the present disclosure can be performed via one or more devices (e.g., one or more controllers) having logic. As used herein, “logic” is an alternative or additional processing resource to execute the actions and/or functions, etc., described herein, which includes hardware (e.g., various forms of transistor logic, application specific integrated circuits (ASICs), etc.), as opposed to computer executable instructions (e.g., software, firmware, etc.) stored in memory and executable by a processor. It is presumed that logic similarly executes instructions for purposes of the embodiments of the present disclosure.

In some embodiments, the application 19 may allow a patient to access predefined programs and/or schedules stored on the IPG or RC. It is further contemplated that the application 19 may include an assistant configured to guide the patient through a reprogramming and/or test procedure. An “assistant” may be a software interface and/or module that provides guidance and tips as a user goes through a series of steps suggested or directed by the application, for example. The application may be launched by the patient and/or automatically in response to some other condition (e.g., receiving an indication that a loss of efficacy is detected). In some embodiments, the application 19 can be executing as a background application.

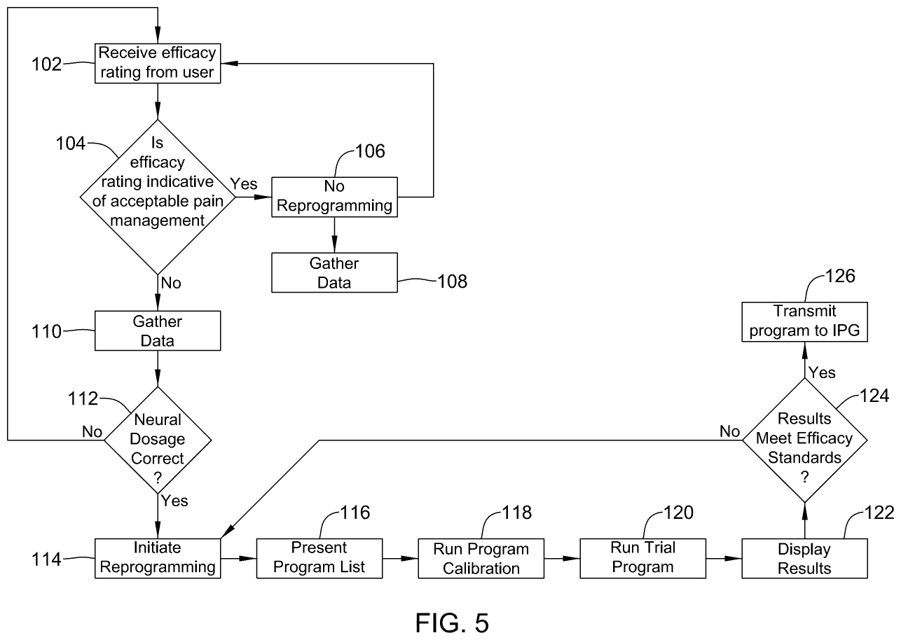

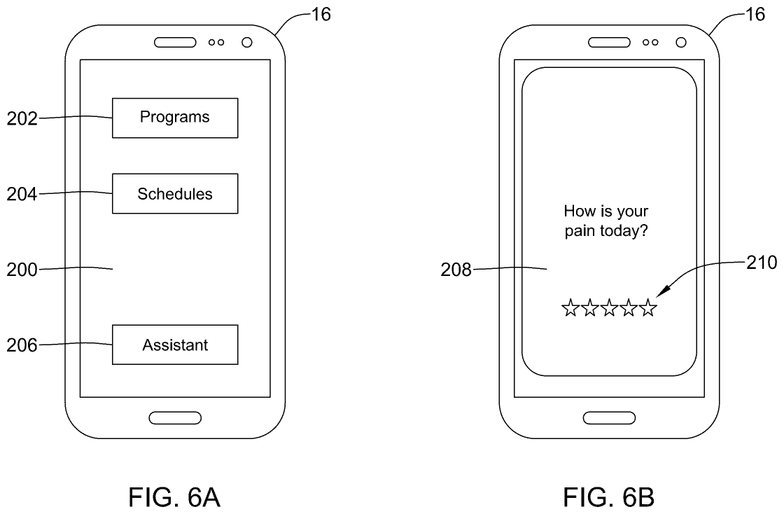

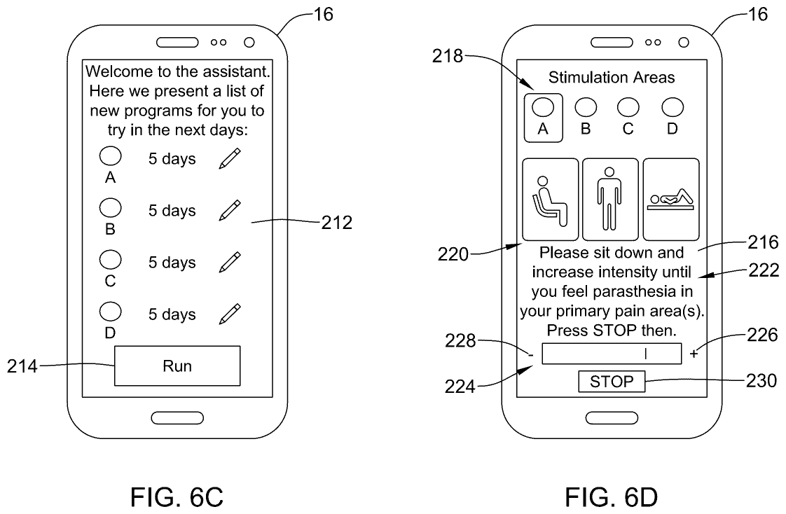

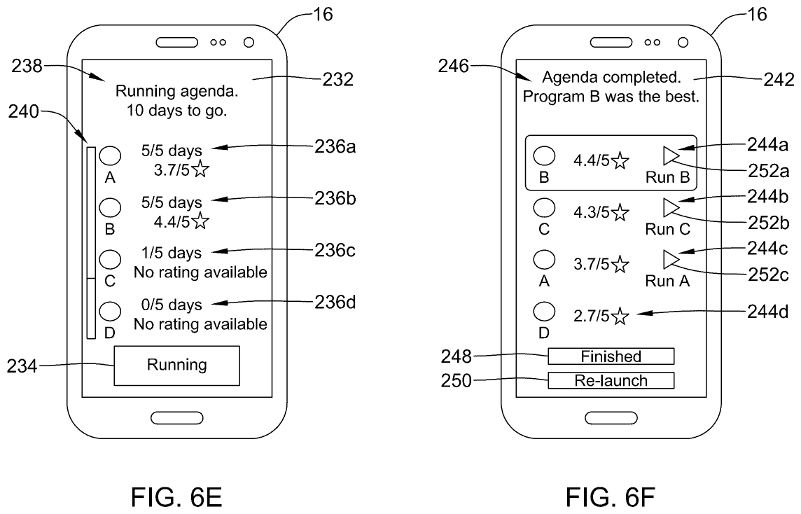

FIG. 5 is an illustrative flow chart of an illustrative method 100 for reprogramming the IPG 14. FIGS. 6A-6F depict illustrative displays that may be presented to the patient via the RC 16 during the reprogramming procedure. The method 100 may be performed by or at the RC 16. Prior to allowing the patient to use the assistant to reprogram the IPG, the RC 16 may be configured to first determine reprogramming is necessary. For example, prior to allowing the patient to access the assistant, the RC 16 may first receive an efficacy rating from the patient, as shown at block 102. Generally, the efficacy rating may be an indication of how well the system 10 is managing their pain and, if it is not working well, how long the system 10 has not been managing their pain.

In some examples, if the patient is having undesired outcomes expressed by low therapy program ratings, a first check before reprogramming and/or testing additional therapy configurations may be as described in commonly assigned U.S. Patent Application Publications Number 2020/0147388, titled TOOLS TO ASSIST SPINAL CORD STIMULATION SELF-REPROGRAMMING, the disclosures of which are hereby incorporated by reference, including confirming that the patient has not programmed the device in a way that is causing undesirable performance. For example, the patient RC 16 may allow amplitude or other adjustments, and allows the patient to turn therapy on and off. If the patient expresses dissatisfaction, the RC 16 may prompt the patient with reminders regarding how much time (neural dose) the patient is to use the system in a given day, for example, as daily overuse can lead to unsatisfactory outcomes over time. The RC 16 may also correlate expressed patient responses with possible corrective actions. For example, if the patient identifies uncomfortable stimulation as occurring, the RC 16 may suggest reducing therapy amplitude, and if the patient expresses that therapy is not providing relief, the RC 16 may suggest increased amplitude. In this way, the RC 16 may provide the patient with useful tips to limit the need for reprogramming and associated program testing. It is contemplated that when the application 19 is launched, the patient may be presented with a home screen. FIG. 6A is an illustrative home screen 200 presented on the launch or opening of the application 19 on the RC 16. The home screen 200 may allow the patient to view or select available programs at icon 202. Further, the home screen 200 may allow the patient to view select available schedules at icon 204. Additionally, the home screen 200 may allow the patient to launch the reprogramming assistant at icon 206 to enter a reprogramming mode. In some examples, the icons 202, 204, 206 may be selected through touching the icon (e.g., as in a touchscreen). The examples described herein are described as a touchscreen. However, it should be understood that if the RC 16 does not include a touchscreen other inputs, may be used to provide user input to the RC 16. For example, instructions may be executed through the selection of an adjacent button (not explicitly shown).

Generally, the reprogramming assistant 206 may guide the patient through a process of trialing preloaded rescue programs that may provide better pain management that the current program. It is contemplated that the reprogramming assistant 206 may be configured to limit a number of layers of screen navigation to provide a concise procedure. While the present disclosure describes the process as patient driven, it is contemplated that the reprogramming process may be completed by another person outside of the clinical setting. For example, a relative or caregiver may assist the patient in the navigation of the reprogramming assistant.

When the patient selects the reprogramming assistant icon 206, the application 19 may be configured to display a notification or pop-up screen 208 asking the patient to indicate how well their pain is being managed that day, as shown in FIG. 6B. The notification 208 may provide user input region 210 for the patient to rate their pain management. It is contemplated that different scales and/or rating systems may be used as desired. In the illustrated embodiment, the RC 16 may display a star rating system where 1 star is indicative of extremely poor pain management and 5 stars is indicative of excellent pain management. In other examples, a numerical score (e.g., a rating from one to ten) may be used. It is contemplated that the rating system may either ask the patient to provide an indication of how well the system is managing the pain or ask the patient to provide an indication of how much pain the are experiencing. It is contemplated that the notification 208 may provide an alphanumerical prompt to the patient to indicate what the rating system is scoring. If the patient indicates that their pain is being poorly managed, the RC 16 may display a second notification asking the patient how long the pain has been poorly managed. The RC 16 may then determine, based on the pain management score and/or duration or poor pain management (if applicable), if the efficacy rating is indicative of acceptable pain management, as shown at block 104.

In some examples, the reprogramming assistant may be launched automatically by the RC 16 (e.g., the reprogramming mode automatically entered) or a notification prompting the patient to launch the reprogramming assistant may be displayed in response to one or more predetermined criteria. For example, the reprogramming assistant may be launched automatically by the RC 16 or a notification prompting the patient to launch the reprogramming assistant may be displayed in response to a detection of a worsening state of the patient, decreased efficacy ratings, etc. For example, the RC 16 may be configured to routinely poll the patient during routine monitoring regarding the efficacy of their pain management. If the efficacy is rated below a predetermined threshold for a predetermined length of time, the reprogramming assistant may be launched automatically by the RC 16 or a notification prompting the patient to launch the reprogramming assistant may be displayed. In such as instance, the application 19 may have already determined that the efficacy rating is not indicative of acceptable pain management (block 104).

If the application 19 determines that the efficacy rating is indicative of acceptable pain management, the application 19 may determine that no reprogramming is required, as shown at block 106. The application 19 may gather data from the patient related to a patient's pain perception, as shown at block 108. Some illustrative data that may be gathered may include an activity level of the patient, a stress level of the patient, the weather, or the like. The application 19 may then return to the beginning of the method 100 and await the next efficacy rating from the patient.

If the application 19 determines that the efficacy rating is not indicative of acceptable pain management, the application 19 may gather data from the patient related to a patient's pain perception, as shown at block 110. Some illustrative data that may be gathered may include an activity level of the patient, a stress level of the patient, the weather, or the like. Subsequent to or concurrently with the data gathering, the application 19 may check the neural dosage the patient is receiving to verify that the dosage is correct, as shown at block 112. If the neural dosage is not correct, the application 19 may adjust the neural dosage to match the programmed amount, and transmit signals to the IPG 14 to control the neural dosage accordingly. Neural dosage may be assessed as a population based metric or at an individual level. Assessment of the neural dosage is described in U.S. Patent Application Publications Number 2019/0046800, titled PARESTHESIA-FREE SPINAL CORD STIMULATION OCCURRING AT LOWER FREQUENCIES AND SWEET SPOT SEARCHING USING PARESTHESIA, the disclosure of which is hereby incorporated by reference. The application 19 may then return to the beginning of the method 100 and await the next efficacy rating from the patient. In some cases, the application 19 may be configured to wait a predetermined length of time (e.g., a day, more than one day, a week, etc.) prior to attempting reprogram the IPG 14 if it is determined that an incorrect neural dosage has been used.

If the neural dosage is correct, the application 19 may initiate reprogramming, as shown at block 114. Initiating reprogramming may include launching or opening the reprogramming assistant. The reprogramming assistant may optionally display a pop-up or notification asking the patient if the paresthesia overlaps the area of pain. The patient feedback may at least partially influence which rescue programs the reprogramming assistant presents to the patient for trial. For example, if the paresthesia overlaps the area of pain, the reprogramming assistant may present at least one trial program that is modified based on the anatomical location. For example, the application may automatically generate one or more new treatment programs based on anatomical locations departing from the reference program. If the paresthesia does not overlap the area of pain, the reprogramming assistant may present at least one trial program that is modified based on the paresthesia rescue location. For example, one or more of the trial programs may be spatially shifted by a predetermined distance from the reference program in different directions.

The application 19 may present the patient with a list of rescue programs to try, as shown at block 116. The rescue programs may be selected from the anatomical based programs or paresthesia-based programs generated after the generation of the reference program. FIG. 6C illustrates an illustrative display screen 212 displaying the list of the trial programs and time frame to the patient. In some cases, the reprogramming trials may be presented as an agenda or list of programs to be accomplished at certain times. Each trial program may be run for a predetermined number of days. In some cases, the predetermined number of days may be five days. However, the predetermined number of days may be less than five days or greater than five days. The application 19 may include a prioritization algorithm which is run at the time the reprogramming assistant is launched to determine which programs to display to the patient. For example, of the programs that are available, only a select number may be presented to the patient for trial. The prioritization algorithm may analyze the available programs based on one or more criteria. In one example, the distance in millimeters of the trial program to the reference program may be a criterion.

Additionally, or alternatively, the prioritization algorithm may take into consideration results from previous trial program runs. For example, if a program has been unsuccessfully previously trialed, that program may be placed lower on the program list. Other conditions and/or criteria that the algorithm may take into consideration include, but are not limited to, how long ago a program was tested (e.g., weeks, months, etc.), if the patient has previously assigned a performance metric to the trial program, has the reference program been updated, has the trial program been recalculated in response to an update to the reference program, and the like.

The algorithm may follow an optimization process and assign the trial programs to be evaluated in a manner which presents the programs most likely to improve pain management to the patient. The algorithm may be configured to take into consideration pain dynamics as a program that was effective in the past may no longer be effective and conversely a program that was ineffective in the past may become effective. It is contemplated that the algorithm may be configured to present four trial programs to the patient. However, fewer than four or more than four trial programs may be presented. The number of trial programs may be selected to provide an increased likelihood of improving pain management over a period of time. The trial programs may be assigned an identification letter (e.g., “A”, “B”, “C”, “D”), an identification number (e.g., 1, 2, 3, 4), or an identification name (e.g., left, right, upper, lower, etc.).

After the trial programs are presented to the patient, the patient may select the “Run” icon 214 on the display of the RC 16 to begin the reprogramming trial procedure. If the RC 16 does not include a touch screen, the patient may select a button associated with the run command. Once the reprogramming trial procedure has been initiated, a calibration mode may be initiated, as shown at block 118. It is noted that the calibration mode may be entered at any time independently of the use of the reprogramming assistant. For example, it may be desired to calibrate all program candidates at a predetermined time interval (e.g., monthly, every other month, every three months, monthly for an initial time period after implantation (e.g., for three to six months) followed by every three months after the initial time period.) These are just some examples. Other calibration intervals may be used as desired.

In the calibration mode, the patient may be presented with a calibration screen 216 on the display of the RC 16, as shown in FIG. 6D. The calibration screen 216 may guide the patient through a calibration process to optimize the program for that particular patient. Referring to FIG. 6D, the application 19 may include a program selection area 218. It is contemplated that the patient may calibrate each program successively upon beginning the reprograming procedure. In other examples, each program may be calibrated immediately before beginning the trial for that particular program. The patient may select the program to be calibrated at the program selection area 218. In the illustrated embodiment, program “A” has been selected. The application 19 may be configured to provide an indication of which program has been selected. This may include highlighting the program name, placing a box or other shape around the program, or the like.

The application 19 may also include a posture selection region 220. The posture selection region 220 may allow the patient to select a particular posture (e.g., sitting, standing, laying down, etc.) to perform the calibration. In some cases, upon completion of the calibration for a particular posture, the application may automatically prompt the patient to perform the calibration in an additional posture until the calibration is completed for each of sitting, standing, and laying down. However, this is not required. In some cases, the posture calibration may be patient selected. In other examples, the calibration mode may not require the calibration process be performed in each of the provided postures. The application 19 may be configured to provide an indication of which posture has been selected. This may include highlighting the posture, placing a box or other shape around the posture, or the like.

The application 19 may also include an instruction display region 222. The instruction display region 222 may include plain language instructions to guide the patient through the calibration process. For example, the patient may be directed to position themselves in the posture to be calibrated. The plain language instructions may change for each posture. In the illustrated embodiments, the sitting posture is being calibrated and as such, the patient is instructed to sit. The instructions may further instruct the patient to increase stimulation until paresthesia is felt in the primary pain area(s). The app may include a stimulation control region 224 include an icon to increase stimulation 226 and an icon to decrease stimulation 228. Alternatively, or additionally, the stimulation may be increased or decreased through a slide bar, or other control mechanisms. Further, the patient may be instructed to select the “STOP” icon 230 when paresthesia is felt. It is contemplated that other words or symbols may be used in place of “STOP” as desired. In some cases, after the calibration process is completed for a particular program and posture, the application 19 may be configured to display a pop-up window including one or more verification questions to determine if the patient ended the calibration process at the appropriate time. For example, the pop-up window may ask the patient how and/or where the stimulating was felt. In one illustrative example, the pop-up window may include a selectable list of answers. For example, if the pop-up window asks the patient where the they felt the stimulation, the selectable responses may include but are not limited to:
(1) In my area of pain (80-100% coverage)
(2) Somewhat in my area of pain (50-80%)
(3) Barely in my area of pain (20-50%)
(4) Not in my area of pain (0-20%)
(5) Undesired area
   It is contemplated that the patient response may be used by the algorithm to determine a therapeutic amplitude and/or to identify whether the program being tested is targeting the correct neural tissue. The calibration procedure may then be continued with the next posture and/or program. In some embodiments, the application 19 may be configured to prompt the patient to perform a recalibration process of the reference program independent of a launch of the reprogramming assistant 206. The recalibration process may be prompted based on a clinical observation (e.g., efficacy rating) or may be prompted at predetermined time intervals (e.g., every so many days, weeks, months, etc. after implantation).

In some examples, if the patient responses indicate that the test program is not delivering therapy to the area of pain, or that paresthesia is in an undesired area, the test program may not advance to the subsequent trial program test at block 120. The patient may be prompted to select a different program by returning to one of blocks 114 or 116, if desired.

Returning to FIG. 5, upon completion of the calibration process, the application 19 may be configured to execute the reprogramming trial period, as shown at block 120. The application 19 may be configured to operate the IPG 14 in accordance with the parameters of each program for a number of consecutive days (e.g., five consecutive days in the illustrated embodiment). For example, to begin the reprogramming trial period, program “A” may be run for five consecutive days. The patient may be prompted to rate or score the pain management provided by the program one or more times each day. In some cases, the frequency at which the application 19 requests patient feedback may be determined, at least in part, on how well pain is being managed. For example, if the patient is consistently providing a good efficacy rating, the application 19 may request feedback less often than if the patient if providing a poor efficacy rating. It is contemplated that the application 19 may be configured to display a pop up or notification asking the patient for a pain rating. The application may average the pain ratings for the program upon the completion of the program. Once a program has been run for the predetermined number of consecutive days the application 19 may automatically begin running the next trial program (e.g., without patient input). However, in some examples, a calibration procedure may be required before beginning subsequent trial programs.

During the trial program run at block 120, the patient may also be allowed to terminate therapy. This may be due to the therapy not working for the patient, or because the therapy is uncomfortable, causing a side effect, or generating paresthesia at an unwanted location. The patient may also terminate therapy for reasons unrelated to efficacy, such as if the patient has been instructed to turn therapy off before engaging in certain activities (driving, certain physical activities, or sleeping, for example), or if the device needs to be recharged. A patient query can be prompted in response to at test therapy being terminated to parse out which of the various reasons therapy may be turned off has arisen.

Programs run during this test process may take the place of programs used by the patient on an ongoing basis, either part-time or full-time. For example, if the patient uses a default or reference program regularly, the test program may be swapped in for use throughout the day whenever the patient has the IPG on and delivering therapy. Alternatively, the test program may be used for a set time period (15 minutes, 30 minutes, 1 hour, etc.), with the system reverting to the default or reference program after the test program is used. Either way, patient queries for feedback may be requested while and promptly after the test program is used. In addition, the patient may be queried for feedback at other times, such as hours after the program is used or the next day.

During the execution of the trial programs, the reprogramming assistant icon 206 may be selected from the home screen 200 of the application 19 to allow the patient to view a status of the reprogramming trials on the display of the RC 16. FIG. 6E illustrates an illustrative status update screen 232. The status update screen 232 may be configured to provide an indication of the reprogramming trial status 234. In the illustrated embodiment, the reprogramming trial is still in process and is indicated as “running.” If the reprogramming trial is complete, the status 234 may indicate “complete.” Other status indicators may be used, as desired. The status update screen 232 may be configured to display a status 236a-d of each program to be trialed. In the illustrated embodiment, the trial of programs “A” and “B” have been completed and the patient provided rating is displayed. Further, the trial of program “C” has begun, but has not been competed and no rating is available. Additionally, the trial of program “D” has not yet begun and no rating is available. The application 19 may optionally be further configured to provide a plain language indication 238 of the reprogramming trial status. In the illustrated embodiment, the plain language indication informs the patient the trial agenda is running and there are 10 days left in the process. It is further contemplated that status update screen 232 may optionally provide a status bar 240, or other non-verbal indications of the trial status.

Returning to FIG. 5, upon completion of the reprogramming trial period, the application 19 may be configured to display the results of the reprogramming trial period to the patient, as shown at block 122. FIG. 6F illustrates an illustrative test summary screen 242. The test summary screen 242 may be configured to display a status 244a-d of each program trialed. The status 244a-d may include the program name and the rating given to the program by the patient. In the illustrated embodiment, the application 19 is configured to arrange the programs in order of efficacy. For example, the program with the highest rating may be displayed at the top of the list. If the program has a rating exceeding a predetermined minimum rating, the program may be selectable by the patient to be used as the program used by the IPG 14. The test summary screen 242 may allow a patient and the application 19 to determine if a program meets a predetermined acceptable pain management standard (block 124 of FIG. 5).

It is contemplated that if the program meets a predetermined acceptable pain management standard, the status 244a-d may include an indication that the program may be used. In the illustrated embodiments, programs “B”, “C”, and “A” each have a rating of greater than 3.5 and each include an icon (e.g., a “play” arrow) 252a-c indicating the program is selectable by the patient. The patient may select the desired program to be used by the IPG 14 going forward. In the illustrated embodiment, the patient has selected program “B” which has the highest pain management rating. The application 19 may be configured to provide an indication of which program has been selected. This may include highlighting the program name, placing a box or other shape around the program, or the like. After selection of the program, the patient may select the “Finished” icon 248 to exit the reprogramming assistant. It is contemplated that other words or symbols may be used in place of “Finished” as desired. Further, upon selection of the “Finished” icon 248, the application 19 may be configured to transmit the program and/or parameters thereof to the IPG 14, as shown at block 126 of FIG. 5, such that the IPG 14 may deliver therapy according the parameters of the selected program.

In some examples, the test summary screen 242 may include a “Re-launch” icon 250. Selection of the re-launch icon 250 may restart the reprogramming trial process. It is contemplated that if none of the selected programs provide a therapy that meets or exceeds the predetermined minimum rating, the process may be repeated with new trial programs. It is contemplated that the patient may repeat the reprogramming trial process until all previously stored rescue programs have been trialed. In some cases, if the reprogramming trial process is unsuccessful one or more times, the application 19 may be configured to display a pop-up or notification to the patient suggesting an alternative course of action. For example, the application 19 may be configured to recommend the patient schedule a visit with their provider.

The application 19 may be configured to track when and/or how often the patient is using the reprogramming assistant 206 to alter the treatment program. It is contemplated that the patient may be precluded from using the reprogramming assistant 206 more than a predetermined number of times over a predetermined length of time. Repeated use of the reprogramming assistant 206 may indicate the pain is poorly controlled and reprogramming of the reference program may be required.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method for reprogramming an implantable medical device, the method comprising entering a reprogramming mode via an assistant in an application running on a patient remote control, wherein the patient remote control includes a user interface, and, while in said reprogramming mode:
displaying on the user interface of the patient remote control one or more trial programs, wherein the one or more trial programs are displayed in response to receiving an initial patient efficacy rating of a current program below a predetermined threshold;
calibrating the one or more trial programs;
successively operating an implantable medical device in accordance with a set of parameters of each trial program of the one or more trial programs over a predetermined time period;
receiving a patient efficacy rating for each trial program of the one or more trial programs; and
determining if at least one trial program of the one or more trial programs meets a predetermined acceptable pain management standard.

2. The method of claim 1, wherein the initial patient efficacy rating is received in response to entering the reprogramming mode.

3. The method of claim 1, wherein the reprogramming mode is entered in response to receiving a patient input at the user interface of the patient remote control.

4. The method of claim 1, wherein the initial patient efficacy rating is received during routine monitoring of the implantable medical device.

5. The method of claim 1, wherein the application is configured to enter the reprogramming mode in response to the initial patient efficacy rating.

6. The method of claim 1, further comprising selecting a trial program that meets the predetermined pain management standard.

7. The method of claim 6, further comprising transmitting the selected trial program to the implantable medical device.

8. The method of claim 1, wherein the one or more trial programs are selected from a plurality of anatomical based programs or paresthesia-based programs based off of a reference program.

9. The method of claim 1, wherein calibrating the one or more trial programs comprises increasing a stimulus until a paresthesia response is felt for each trial program of the one or more trial programs.

10. The method of claim 9, wherein calibrating the one or more trial programs is performed in one or more postures.

11. The method of claim 9, wherein calibrating the one or more trial programs determines a therapeutic amplitude for each trial program of the one or more trial programs.

12. The method of claim 1, wherein if at least one trial program of the one or more trial programs fails to meet a predetermined acceptable pain management standard, one or more additional trial programs are presented.

13. A non-transitory computer-readable medium having instructions stored thereon that when executed by a patient remote control are configured to:
receive an initial patient efficacy rating of a current program;
display one or more trial programs;
guide a patient through a calibration process of the one or more trial programs;
successively operate an implantable medical device in accordance with a set of parameters of each trial program of the one or more trial programs over a predetermined time period;
receive a patient efficacy rating for each trial program of the one or more trial programs; and
determine if at least one trial program of the one or more trial programs meets a predetermined acceptable pain management standard.

14. The non-transitory computer readable medium of claim 13, wherein the instructions, when executed by the patient remote control, are configured to determine if at least one trial program of the one or more trial programs meets the predetermined acceptable pain management standard and, if not, to present one or more additional trial programs.

15. The non-transitory computer readable medium of claim 14, further configured to transmit the selected trial program to an implantable medical device.

16. A remote control configured for reprogramming an implantable medical device, remote control comprising a memory, a user interface for receiving inputs from and providing instructions or information to a patient, and a processor including a communication circuit for communicating with the implantable medical device, the memory storing in non-transitory manner instructions for performing the following reprogramming mode:
receiving, at the user interface, an initial patient efficacy rating of a current program below a predetermined threshold;
in response to the initial patient efficacy rating being below the predetermined threshold, displaying on the user interface of the patient remote control one or more trial programs;
calibrating the one or more trial programs;
instructing, via the communications circuit, the implantable medical device to deliver therapy to the patient in accordance with a set of parameters of each trial program of the one or more trial programs over a predetermined time period;
receiving, at the user interface, a trial patient efficacy rating for each trial program of the one or more trial programs; and
determining if at least one trial program of the one or more trial programs meets a predetermined acceptable pain management standard.

17. The remote control of claim 16, wherein the reprogramming mode is entered in response to receiving a patient input at the user interface of the patient remote control.

18. The remote control of claim 17, wherein the reprogramming mode is entered in response to the initial patient efficacy rating.

19. The remote control of claim 16, further comprising presenting, via the user interface, a plurality of trial programs to select from, including a plurality of anatomical based programs or paresthesia-based programs based off of a reference program.

20. The remote control of claim 16, wherein the instructions are configured to calibrate the one or more trial programs by increasing a stimulus until a paresthesia response is felt for each trial program of the one or more trial programs.
